(19) United States
(12) Patent Application Publication
(10) Pub. No.: US 2025/0074854 A1
(43) Pub. Date: Mar. 6, 2025

(54) PROCESS FOR THE SELECTIVE CATALYTIC HYDROGENATION OF DIENONES

(71) Applicants: BASF SE, Ludwigshafen am Rhein (DE); University of Alberta, Edmonton (CA)

(72) Inventors: Mathias SCHELWIES, Ludwigshafen am Rhein (DE); Rylan LUNDGREN, Edmonton (CA); Rocco PACIELLO, Ludwigshafen am Rhein (DE); Carlos LIZANDARA PUEYO, Fremont, CA (US)

(21) Appl. No.: 18/728,126

(22) PCT Filed: Jan. 12, 2023
(86) PCT No.: PCT/US2023/010712

§ 371 Date: Jul. 11, 2024

Related U.S. Application Data
(60) Provisional application No. 63/299,135, filed on Jan. 13, 2022.

(51) Int. Cl. C07C 45/62

(52) U.S. Cl. CPC C07C 45/62; C07C 2523/46; C07C 2531/18; C07C 2531/20

(57) ABSTRACT

The present disclosure provides a catalyst system that is capable of selectively hydrogenating (2,3)/(4,5) and (2,3)/(5,6) dienones with hydrogen gas. Specifically, the present disclosure provides catalysts capable of providing high selectivity for the reduction even in the absence of catalyst poisons such as pyridine, pyrazine, quinoline, and quinoxaline

The present invention provides a method for selective hydrogenation of (2,3)/(4,5) unsaturated dienones using a rhodium or ruthenium complex without the need for nitrogen-containing additives such as pyridine, pyrazine, quinoline, and quinoxaline.

### BACKGROUND

Technical Pseudoionone (PI) is readily available from producers of Vitamin A, as it is one of the intermediates towards Vitamin A (see Tetrahedron 2016, 72, 1645-1652). A direct hydrogenation enables the manufacture of valuable intermediate geranylacetone form PI. Geranylacetone is classically made as E/Z-mix from linalool as described in H. Surburg and J. Panten, Common Fragrance and Flavor Materials, 4th Ed., Wiley-VCH, Weinheim 2016. Technical PI is produced and used as a mix of the possible stereoisomers in any ratio; namely 3E/5E-PI, 3E/5Z-PI, 3Z/5E-PI and 3Z/5Z-PI. The 3E/5E-PI isomer is the main isomer of technical PI. Geranylacetone from PI by hydrogenation is produced as E/Z-mix, as shown in Scheme 1 below.

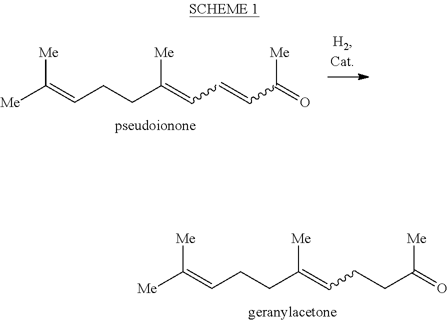 [SMILES: CC(=O)C=CC=C(C)CCC=C(C)C ; CC(=O)CCC=C(C)CCC=C(C)C]

Processes for the selective monohydrogenation of (2,3)/(4,5) unsaturated dienones to the corresponding (4,5) unsaturated enones are known in the literature; however, very few methods are reported that can be run with the efficiency necessary for the use in chemical industry.

The hydrogenation of β-ionone to the corresponding dihydroionone has been described with a heterogeneous copper-catalyst under hydrogen atmosphere at 1 bar and 90° C. (Journal of Molecular Catalysis 74, 1992, 267-74). A similar process is described in Catalysts 2020, 10, 515, wherein the hydrogenation of β-ionone was run at 1 bar of hydrogen and 90° C. Despite these reports, the direct hydrogenation of pseudoionone (PI) to geranylacetone (GAC) with a homogeneous catalyst has only recently been described.

Pseudoionone (PI) is more challenging as substrate for a selective monohydrogenation in (2,3)-position compared to β-ionone, since β-ionone is tetra-substituted at the second double bond while pseudoionone (PI) is trisubstituted (the substrate-dependent reactivity difference of the two double bonds in each molecule is larger for β-ionone than for pseudoionone, making a selective monohydrogenation more difficult for pseudoionone). In Journal of Molecular Catalysis 74, 1992, 267-74 it is additionally noted that for certain substrates, such as β-ionone, the yield drops for the selective monohydrogenation since the second double bond in the molecule isomerizes. Thus, heterogeneous catalysts can lead to isomerization of additional double bonds and therefore cannot be used for the hydrogenation of pseudoionone (PI) to geranylacetone (GAC).

A rhodium-catalyzed selective reduction of (2,3)/(4,5) unsaturated dienones such as β-ionone using Et₃SiH to the corresponding dihydroionone is reported in Organometallics 101982, 1390-1399. However, Et₃SiH is less atom economical than hydrogen.

CN105218339 describes conditions for the selective hydrogenation of methylheptyl dienone to methyl heptanone using Pd (acac)₂/1,2-bis(diphenylphosphino)ethane or Rh(PPh₃)₃Cl/1,2-bis(diphenylphosphino)ethane as catalyst.

WO2012/150053 reports a homogeneous rhodium catalyst system for the selective hydrogenation of (2,3)/(4,5) unsaturated aldehydes to obtain the corresponding (4,5) unsaturated aldehydes. The patent does not mention the application of such a catalyst system for the selective hydrogenation of (2,3)/(4,5) unsaturated dienones.

CN201811560479.9 describes a method for the selective hydrogenation of (2,3)/(4,5) unsaturated dienones using a Ru-complex in the presence of a catalyst poison. It is noted that the hydrogenation of pseudoionone (PI) to geranylacetone (GAC) with hydrogen only proceeds with high selectivity in the presence of nitrogen-containing additives such as pyridine, pyrazine, quinoline, and quinoxaline.

A reduction of (2,3)/(4,5) unsaturated esters is reported in Angew Chem. Int. Ed., 2019, 58, 12246-51, using a Rh-catalyst with formic acid as the reductant as opposed to hydrogen. However, formic acid is less atom economical than hydrogen. In the published cases, the Z-(3,4) unsaturated esters are obtained.

Thus, no system has thus far been described to catalyze the hydrogenation pseudoionone (PI) to geranylacetone (GAC) with high selectivity using hydrogen gas in the absence of pyridine, pyrazine, quinoline, and quinoxaline. The inventors have surprisingly found a catalyst system capable of hydrogenating PI in the absence of a catalyst poison, while retaining high selectivity.

### SUMMARY

The present disclosure provides a method for selective hydrogenation of dienones. Specifically, the present disclosure provides a method comprising: 1) combining a dienone with one or more solvents; 2) adding a catalyst to the mixture of dienone and solvent to provide a reaction mixture; and 3) mixing the reaction mixture under an atmosphere comprising hydrogen (H₂). The atmosphere may also include carbon monoxide (CO). The catalyst may comprise one or more transition metals, such as rhodium and ruthenium, for example. The catalyst may further comprise one or more ligands, such as mono- or bis-phosphines, for example. The reaction may be performed in the absence of a catalyst poison such as pyridine, pyrazine, quinoline, or quinoxaline, while still retaining high selectivity.

### DETAILED DESCRIPTION

### 1. Definitions

In the definitions of the variables given in the formulas above and below, collective terms are used which are generally representative of the respective substituents. The meaning Cₙ- to Cₘ- indicates the respective possible number of carbon atoms in the particular substituents or substituent moiety.

In the context of the present invention, the expression “alkyl” comprises unbranched or branched alkyl groups having 1 to 4, 6, 12 or 25 carbon atoms. These include, for example, C₁- to C₆-alkyl, such as methyl, ethyl, propyl, isopropyl, n-butyl, isobutyl, sec-butyl, tert-butyl, n-pentyl, 2-pentyl, 2-methylbutyl, 3-methylbutyl, 1,2-dimethylpropyl, 1,1-dimethylpropyl, 2,2-dimethylpropyl, 1-ethylpropyl, n-hexyl, 2-hexyl, 2-methylpentyl, 3-methylpentyl, 4-methylpentyl, 1,2-dimethylbutyl, 1,3-dimethylbutyl, 2,3-dimethylbutyl, 1,1-dimethylbutyl, 2,2-dimethylbutyl, 3,3-dimethylbutyl, 1,1,2-trimethylpropyl, 1,2,2-trimethylpropyl, 1-ethylbutyl, 2-ethylbutyl, 1-ethyl-2-methylpropyl and the like.

In the context of the present invention, the expression “cycloalkyl” comprises cyclic, saturated hydrocarbon groups having 3 to 6, 12 or 25 carbon ring members, e.g. C₃-C₈-cycloalkyl such as cyclopropyl, cyclobutyl, cyclopentyl, cyclohexyl, cycloheptyl and cyclooctyl, or C₇-C₁₂-bicycloalkyl.

In the context of the present invention, the expression “alkoxy” is an alkyl group having 1 to 6 carbon atoms bonded via an oxygen, e.g. C₁- to C₆-alkoxy, such as methoxy, ethoxy, n-propoxy, 1-methylethoxy, butoxy, 1-methylpropoxy, 2-methylpropoxy, 1,1-dimethylethoxy, pentoxy, 1-methylbutoxy, 2-methylbutoxy, 3-methylbutoxy, 1,1-dimethylpropoxy, 1,2-dimethylpropoxy, 2,2-dimethylpropoxy, 1-ethylpropoxy, hexoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy or 1-ethyl-2-methylpropoxy.

In the context of the present invention, the expression “alkenyl” comprises unbranched or branched hydrocarbon radicals having 2 to 4, 6, 12 or 25 carbon atoms which comprise at least one double bond, for example 1, 2, 3 or 4 double bonds. These include, for example, C₂-C₆-alkenyl such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl.

In the context of the present invention, the expression “alkylene” refers to divalent hydrocarbon radicals having 2 to 25 carbon atoms. The divalent hydrocarbon radicals can be unbranched or branched. These include, for example, C₂-C₁₆-alkylene groups, such as 1,4-butylene, 1,5-pentylene, 2-methyl-1,4-butylene, 1,6-hexylene, 2-methyl-1,5-pentylene, 3-methyl-1,5-pentylene, 1,7-heptylene, 2-methyl-1,6-hexylene, 3-methyl-1,6-hexylene, 2-ethyl-1,5-pentylene, 3-ethyl-1,5-pentylene, 2,3-dimethyl-1,5-pentylene, 2,4-dimethyl-1,5-pentylene, 1,8-octylene, 2-methyl-1,7-heptylene, 3-methyl-1,7-heptylene, 4-methyl-1,7-heptylene, 2-ethyl-1,6-hexylene, 3-ethyl-1,6-hexylene, 2,3-dimethyl-1,6-hexylene, 2,4-dimethyl-1,6-hexylene, 1,9-nonylene, 2-methyl-1,8-octylene, 3-methyl-1,8-octylene, 4-methyl-1,8-octylene, 2-ethyl-1,7-heptylene, 3-ethyl-1,7-heptylene, 1,10-decylene, 2-methyl-1,9-nonylene, 3-methyl-1,9-nonylene, 4-methyl-1,9-nonylene, 5-methyl-1,9-nonylene, 1,11-undecylene, 2-methyl-1,10-decylene, 3-methyl-1,10-decylene, 5-methyl-1,10-decylene, 1,12-dodecylene, 1,13-tridecylene, 1,14-tetradecylene, 1,15-pentadecylene, 1,16-hexadecylene and the like.

In the mono- or poly-branched or substituted alkylene groups, the carbon atom at the branching point or the carbon atoms at the respective branching points or the carbon atoms carrying a substituent can have, independently of one another, a R- or S-configuration or both configurations in equal or different proportions.

In the context of the present invention, the expression “alkenylene” refers to divalent hydrocarbon radicals having 2 to 25 carbon atoms, which can be unbranched or branched, where the main chain has one or more double bonds, for example 1, 2 or 3 double bonds. These include, for example, C₂- to C₁₈-alkenylene groups, such as ethylene, propylene, 1-, 2-butylene, 1-, 2-pentylene, 1-, 2-, 3-hexylene, 1,3-hexadienylene, 1,4-hexadienylene, 1-, 2-, 3-heptylene, 1,3-heptadienylene, 1,4-heptydienylene, 2,4-heptadienylene, 1-, 2-, 3-octenylene, 1,3-octadienylene, 1,4-octadienylene, 2,4-octadienylene, 1-, 2-, 3-nonenylene, 1-, 2-, 3-, 4-, 5-decenylene, 1-, 2-, 3-, 4-, 5-undecenylene, 2-, 3-, 4-, 5-, 6-dodecenylene, 2,4-dodecadienylene, 2,5-dodecadienylene, 2,6-dodecadienylene, 3-, 4-, 5-, 6-tridecenylene, 2,5-tridecadienylene, 4,7-tridecadienylene, 5,8-tridecadienylene, 4-, 5-, 6-, 7-tetradecenylene, 2,5-tetradecadienylene, 4,7-tetradecadienylene, 5,8-tetradecadienylene, 4-, 5-, 6-, 7-pentadecenylene, 2,5-pentadecadienylene, 4,7-pentadecadienylene, 5,8-pentadecadienylene, 1,4,7-pentadecatrienylene, 4,7,11-pentadecatrienylene, 4,6,8-pentadecatrienylene, 4-, 5-, 6-, 7-, 8-hexadecenylene, 2,5-hexadecadienylene, 4,7-hexadecadienylene, 5,8-hexadecadienylene, 2,5,8-hexadecatrienylene, 4,8,11-hexadecatrienylene, 5,7,9-hexadecatrienylene, 5-, 6-, 7-, 8-heptadecenylene, 2,5-heptadecadienylene, 4,7-heptadecadienylene, 5,8-heptadecadienylene, 5-, 6-, 7-, 8-, 9-octadecenylene, 2,5-octadecadienylene, 4,7-octadecadienylene, 5,8-octadecadienylene and the like.

The double bonds in the alkenylene groups can be present independently of one another in the E and also in the Z configuration or as a mixture of both configurations.

In the context of the present invention, the expression “halogen” comprises fluorine, chlorine, bromine and iodine, preferably fluorine, chlorine or bromine. In the context of the present invention, the expression “aryl” comprises a mono-to

30 trinuclear aromatic ring system comprising 6 to 14 carbon ring members. These include, for example, C₆- to C₁₀-aryl, such as phenyl or naphthyl.

In the context of the present invention, the expression “hetaryl” comprises mono-to trinuclear aromatic ring system comprising 6 to 14 carbon ring members, where one or more, for example 1, 2, 3, 4, 5 or 6, carbon atoms are substituted by a nitrogen, oxygen and/or sulfur atom. These include, for example, C₃- to C₉-hetaryl groups, such as 2-furyl, 3-furyl, 2-thienyl, 3-thienyl, 2-pyrrolyl, 3-pyrrolyl, 3-isoxazolyl, 4-isoxazolyl, 5-isoxazolyl, 3-isothiazolyl, 4-isothiazolyl, 5-isothiazolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 2-imidazolyl, 4-imidazolyl, 1,2,4-oxadiazol-3-yl, 1,2,4-oxadiazol-5-yl, 1,2,4-thiadiazol-3-yl, 1,2,4-thiadiazol-5-yl, 1,2,4-triazol-3-yl, 1,3,4-oxadiazol-2-yl, 1,3,4-thiadiazol-2-yl, 1,3,4-triazol-2-yl, 2-pyridinyl, 3-pyridinyl, 4-pyridinyl, 3-pyridazinyl, 4-pyridazinyl, 2-pyrimidinyl, 4-pyrimidinyl, 5-pyrimidinyl, 2-pyrazinyl, 1,3,5-triazin-2-yl, 1,2,4-triazin-3-yl, 2-indolyl, 3-indolyl, 4-indolyl, 5-indolyl, 6-indolyl, 7-indolyl and the like.

In the context of the present invention, the expression “aralkyl” comprises a mono-to dinuclear aromatic ring system, comprising 6 to 10 carbon ring members, bonded via an unbranched or branched C₁- to C₆-alkyl group. These include, for example, C₇- to C₁₂-aralkyl, such as phenylmethyl, 1-phenylethyl, 2-phenylethyl, 1-phenylpropyl, 2-phenylpropyl, 3-phenylpropyl and the like.

In the context of the present invention, the expression “aralkyl” comprises mono-to dinuclear aromatic ring systems comprising 6 to 10 carbon ring members which is substituted with one or more, for example 1, 2 or 3, unbranched or branched C₁- to C₆-alkyl radicals. These include e.g. C₇- to C₁₂-alkylaryl, such as 1-methylphenyl, 2-methylphenyl, 3-methylphenyl, 1-ethylphenyl, 2-ethylphenyl, 3-ethylphenyl, 1-propylphenyl, 2-propylphenyl, 3-propylphenyl, 1-isopropylphenyl, 2-isopropylphenyl, 3-isopropylphenyl, 1-butylphenyl, 2-butylphenyl, 3-butylphenyl, 1-isobutylphenyl, 2-isobutylphenyl, 3-iso-butylphenyl, 1-sec-butylphenyl, 2-sec-butylphenyl, 3-sec-butylphenyl, 1-tert-butylphenyl, 2-tert-butylphenyl, 3-tert-butylphenyl, 1-(1-pentenyl)phenyl, 2-(1-pentenyl)phenyl, 3-(1-pentenyl)phenyl, 1-(2-pentenyl)phenyl, 2-(2-pentenyl)phenyl, 3-(2-pentenyl)phenyl, 1-(3-pentenyl)phenyl, 2-(3-pentenyl)phenyl, 3-(3pentenyl)phenyl, 1-(1-(2-methylbutyl))phenyl, 2-(1-(2-methylbutyl))phenyl, 3-(1-(2-methylbutyl))phenyl, 1-(2-(2-methylbutyl))phenyl, 2-(2-(2-methylbutyl))phenyl, 3-(2-(2-methylbutyl))phenyl, 1-(3-(2-methylbutyl))phenyl, 2-(3-(2-methylbutyl))phenyl, 3-(3-(2-methylbutyl))phenyl, 1-(4-(2-methylbutyl))phenyl, 2-(4-(2-methylbutyl))phenyl, 3-(4-(2-methylbutyl))phenyl, 1-(1-(2,2-dimethylpropyl))phenyl, 2-(1-(2,2-dimethylpropyl))phenyl, 3-(1-(2,2-dimethylpropyl)phenyl, 1-(1-hexenyl)phenyl, 2-(1-hexenyl)phenyl, 3-(1-hexenyl)phenyl, 1-(2-hexenyl)phenyl, 2-(2-hexenyl)phenyl, 3-(2-hexenyl)phenyl, 1-(3-hexenyl)phenyl, 2-(3-hexenyl)phenyl, 3-(3-hexenyl)phenyl, 1-(1-(2-methylpentenyl))phenyl, 2-(1-(2-methylpentenyl))phenyl, 3-(1-(2-methylpentenyl))phenyl, 1-(2-(2-methylpentenyl))phenyl, 2-(2-(2-methylpentenyl))phenyl, 3-(2-(2-methylpentenyl))phenyl, 1-(3-(2-methylpentenyl))phenyl, 2-(3-(2-methylpentenyl))phenyl, 3-(3-(2-methylpentenyl))phenyl, 1-(4-(2-methylpentenyl))phenyl, 2-(4-(2-methylpentenyl))phenyl, 3-(4-(2-methylpentenyl))phenyl, 1-(5-(2-methylpentenyl))phenyl, 2-(5-(2-methylpentenyl))phenyl, 3-(5-(2-methylpentenyl))phenyl, 1-(1-(2,2-dimethylbutenyl))phenyl, 2-(1-(2,2-dimethylbutenyl))phenyl, 3-(1-(2,2-dimethylbutenyl))phenyl, 1-(3-(2,2-dimethylbutenyl))phenyl, 2-(3-(2,2-dimethylbutenyl))phenyl, 3-(3-(2,2-dimethylbutenyl))phenyl, 1-(4-(2,2-dimethyl-butenyl))phenyl, 2-(4-(2,2-dimethylbutenyl))phenyl, 3-(4-(2,2-dimethylbutenyl))phenyl and the like.

### 2. Catalysts

The present disclosure provides a catalyst system that is capable of selectively hydrogenating dienones with hydrogen gas. Suitable dienones may include (2,3)/(4,5) and (2,3)/(5,6) dienones, such as pseudoionone, β-ionones, 6-methyl-3,5-heptadien-2-one, and α-ionone, for example. Specifically, the present disclosure provides catalysts capable of providing high selectivity for the reduction. Surprisingly, it has been found that the catalyst systems of the present disclosure are capable of catalyzing the hydrogenation under hydrogen gas in the absence of a catalyst poison, such as pyridine, pyrazine, quinoline, and quinoxaline, while achieving high selectivity. This may be particularly desirable as these catalyst poisons must be removed following the reaction. Thus, the method of the present disclosure allows for high selectivity, greater atom economy, and simpler purification.

The catalyst may comprise one or more transition metals and one or more ligands.

The one or more transition metals may be selected from the group comprising ruthenium, rhodium, platinum, palladium, and nickel.

The catalyst may be formed by reacting a transition metal containing precursor with a ligand (and possibly an additional reagent such as for example H₂, CO, MeOH, reducing agent) in any ratio to form a metal-ligand-complex. For example when the ligand is a neutral bidentate bisphosphine ligand, the metal-ligand complex may be of one of the following forms: (L)M(CO)X; (L)M(CO)₂X; (L)M(CO)XY; (L)M(CO)XYZ. For example when the ligand is a monodentate phosphine ligand, the metal-ligand complex may be of one of the following forms: (L)₂M(CO)X; (L)₂M(CO)₂X; (L)₃M(CO)X; (L)₂M(CO)XY; (L)₂M(CO)XY; (L)₂M(CO)XYZ. X, Y and Z are each independently anionic monodentate ligands, for example H, Cl, Br, OAc, OH, acac, OMe, OEt, or OAlkyl. Suitable metal containing precursors include Rh(CO)₂acac, Rh(III) acetate, or [Ru(COD)(2-methylallyl)₂].

Other suitable metal containing precursors include rhodium or ruthenium metal complexes. Suitable rhodium compounds are in particular those which are soluble in the selected reaction medium, such as, for example, rhodium (0), rhodium(I), rhodium(II) and rhodium(III) salts such as e.g. rhodium(III) chloride, rhodium(III) bromide, rhodium(III) nitrate, rhodium(III) sulfate, rhodium(II) or rhodium(III) oxide, rhodium(II) or rhodium(III) acetate, rhodium(II) or rhodium(III) carboxylate, Rh(acac)₃, [Rh(cod)Cl]₂, [Rh(cod)₂]BF₄, Rh₂(OAc)₄, bis(ethylene)rhodium(I)acac, Rh(CO)₂acac, [Rh(cod)OH]₂, [Rh(cod)OMe]₂, Rh₄(CO)₁₂ or Rh₆(CO)₁₆, where “acac” is an acetylacetonate ligand, “cod” is a cyclooctadiene ligand and “OAc” is an acetate ligand.

Further catalysts rhodium compounds may include carbonyl-containing rhodium compounds of the type L₂Rh (CO) H (with L=Monodentate Phosphine), L₃Rh(CO)H (with L=Monodentate Phosphine) or LRh(CO)H (with L=Bidentate Phosphine) or LRh(CO)₂H (with L=Bidentate Phosphine). Such complexes have been used in olefin hydrogenation as described by Wilkinson et al in J. Chem. Soc. (A) 1968, 2665-2671 (using (PPh₃)₃Rh(CO)H) or Breit et al in Tetrahedron Letters 2005, 6171-6179 (using (PPh₃)₃Rh(CO)H) or by Delongchamps et al in Can. J. Chem 1990, 2137-2143 or by Jäkel et al in Adv. Synth. Catal. 2008, 2708-2714 (using (Chiraphos)Rh(CO)₂H) but not in the selective hydrogenation of unsaturated dienones. Such carbonyl compounds can be used as isolated complexes or prepared by catalyst preformation.

Suitable ruthenium compounds are in particular those which are soluble in the selected reaction medium, such as, for example, ruthenium(0), ruthenium(I), ruthenium(II) and ruthenium (III) salts such as e.g. [Ru(p-cymene)Cl₂]₂, [Ru(CO)₄(ethylene)], [Ru(COD)(OAc)₂], [Ru(CO)₂Cl₂]ₙ, [Ru(CO)₃Cl₂]₂, [RuCl₃*H₂O], [Ru(acetylacetonate)₃], [Ru(benzene)Cl₂]ₙ, [Ru(COD)(2-methylallyl)₂], [Ru(DMSO)₄Cl₂], [Ru (PPh₃)₃(CO)(H) Cl], [Ru(PPh₃)₃(CO)Cl₂], [Ru(PPh₃)₃(CO)(H)₂], [Ru(PPh₃)₃Cl₂], [Ru(COD)Cl₂]₂, [Ru(pentamethylcyclo-pentadienyl)(COD)Cl], [Ru₃(CO)₁₂], for example.

The ligand may comprise one or more bisphosphines, one or more monophosphines, or a combination thereof. The ligand may be chosen from the group comprising 4,5-bis(dipenylphosphino)-9,9-dimethylxanthene (xantphos), bis[(2-diphenylphosphino)phenyl] ether (DPEphos), bis(diphenylphosphino)methane (dppm), 1,2-bis(diphenylphosphino)ethane (dppe), 1,3-bis(diphenylphosphino)propane (dppp), 1,4-bis(diphenylphosphino)butane (dppb), 1,1′-bis(diphenylphosphino)ferrocene (dppf), 2,2′-bis(diphenylphosphino)-1,1′-binaphthyl (BINAP), (4,4,4′,4′,6,6′-hexamethyl-3,3′,4,4′-tetrahydro-2,2′-spirobi[[1] benzopyran]-8,8′-diyl)bis(diphenylphosphane) (SPANPhos), triphenyl phosphite (P(OPh)₃), 6,6′-[(3,3′-di-tert-butyl-5,5′-dimethoxy-[1,1′-biphenyl]-2,2′-diyl)bis(oxy)]bis(6H-dibenzo[d,f][1,3,2]dioxaphosphepine) (BiPhePhos), trimethyl phosphite (P(OMe)₃), triethyl phosphite (P(OEt)₃), (3,5-dioxa-4-phosphacyclohepta[2,1-a:3,4-a′] dinaphthalene-4-yl)dimethylamine (MonoPhos), (R,R) Chiraphos, (S,S) Chiraphos, 2-dicyclohexylphosphino-2′,6′-dimethoxybiphenyl (SPhos), triphenylphosphine (PPh₃), tris (4-methoxyphenyl)phosphane, tris(3,5-bis(trifluoromethyl)phenyl)phosphane, 1,1′-bis(diisopropylphosphino)ferrocene (dippf), and methyldiphenylphosphane. The structures of the ligands are shown below, in which “Me” is to be understood as meaning methyl, “Ph” phenyl, “Cy” cyclohexyl, and ⁱPr isopropanol.

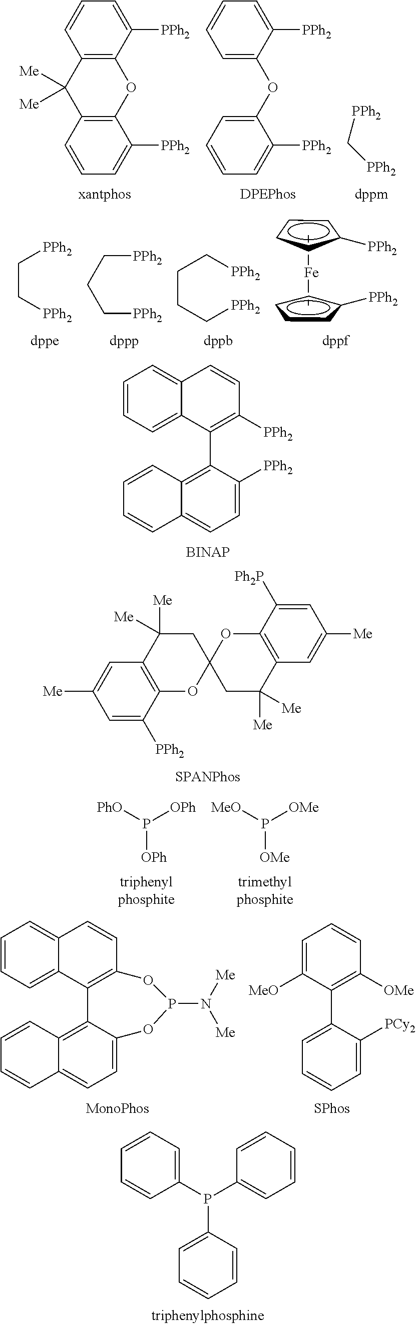

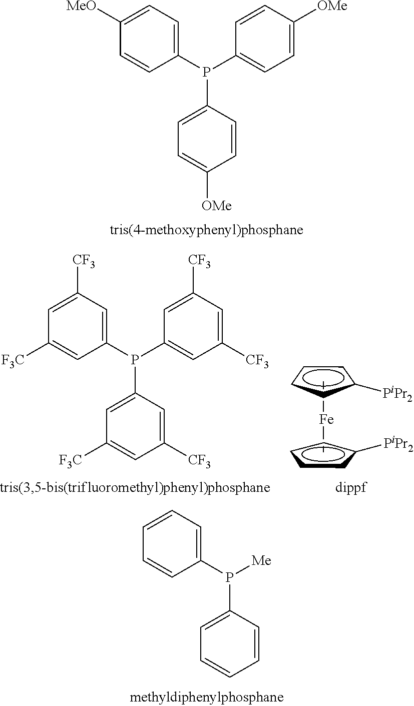

The transition metal complex may be present in the reaction in an amount of about 0.01 mol % or greater, about 0.05 mol % or greater, about 0.1 mol % or greater, about 0.2 mol % or greater, about 0.3 mol % or greater, about 0.4 mol % or less, about 0.5 mol % or less, about 0.6 mol % or less, about 0.7 mol % or less, about 0.8 mol % or less, about 0.9 mol % or less, about 1.0 mol % or less, or any value encompassed by these endpoints.

The molar ratio of the ligand (that can be a monodentate or a bidentate phosphine ligand) to the metal may be about 0.9:1 or greater, about 1.0:1 or greater, about 1.5:1 or greater, about 2.0:1 or greater, about 2.5:1 or greater, about 3.0:1 or greater, about 5:1 or greater, about 10:1 or greater, about 20:1 or greater, about 30:1 or greater, about 40:1 or greater, about 50:1 or less, about 60:1 or less, about 70:1 or less, about 80:1 or less, about 90:1 or less, about 100:1 or less, or any value encompassed by these endpoints. In a chemical process, the ligand can be oxidized or partially oxidized over time, for example by oxidizing contamination in the feed. Also the catalyst can be decomposed by base or thermal stress over time. Thus the optimal ratio of the ligand to metal is dependent on various parameters and can be different in different setups.

The ligand may be present in the reaction in an amount of about 0.01 mol % or greater, about 0.05 mol % or greater, about 0.1 mol % or greater, about 0.5 mol % or greater, about 1.0 mol % or greater, about 2.0 mol % or greater, about 3.0 mol % or greater, about 4.0 mol % or less, about 5.0 mol % or less, about 6.0 mol % or less, about 7.0 mol % or less, about 8.0 mol % or less, about 9.0 mol % or less, about 10.0 mol % or less, or any value encompassed by these endpoints.

The reaction may be performed in the presence of a base, such as Na₂CO₃, NaOMe, NaOEt, or trialkyl amines such as triethylamine, ethyldiisopropyl amine, and triisopropylamine, for example.

### 3. Reaction Conditions

As noted above, the above catalysts are capable of providing high selectivity for reduction of (2,3)/(4,5) and (2,3)/(5,6) dienones, even in the absence of pyridine, pyrazine, quinolone, and quinoxaline. Suitable dienones may include (2,3)/(4,5) and (2,3)/(5,6) dienones, such as pseudoionone, β-ionones, 6-methyl-2-hept-5-en-2-one, and α-ionone, for example.

In one embodiment, suitable substrates for the reaction may include (2,3)/(4,5) dienones of Formula I, shown below,

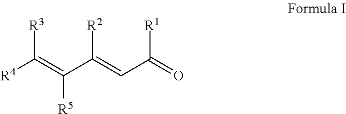

wherein R¹ is C₁-C₆ alkyl, C₁-C₆ alkoxy, or a bond to form an optionally substituted 5- or 6-membered ring with R²; R² is hydrogen, C₁-C₆ alkyl, or a bond to form an optionally substituted 5- or 6-membered ring with R¹; R³ is hydrogen, C₁-C₆ alkyl, C₁-C₁₀ alkenyl, or aryl; R⁴ is hydrogen, C₁-C₆ alkyl, C₁-C₁₀ alkenyl, aryl, or a bond to form an optionally substituted 5- or 6-membered ring with R⁵; and R⁵ is hydrogen, C₁-C₆ alkyl, C₁-C₁₀ alkenyl, aryl, or a bond to form an optionally substituted 5- or 6-membered ring with R⁴.

In another embodiment, suitable substrates for the reaction may include (2,3)/(5,6) dienones of Formula II, shown below.

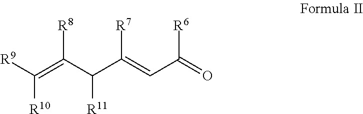

wherein R⁶ is C₁-C₆ alkyl, or C₁-C₆ alkoxy; R⁷ is hydrogen, or C₁-C₆ alkyl; R⁸ is hydrogen, C₁-C₆ alkyl, C₁-C₁₀ alkenyl, or aryl; R⁹ is hydrogen, C₁-C₆ alkyl, C₁-C₁₀ alkenyl, or aryl; R¹⁰ is hydrogen, C₁-C₆ alkyl, C₁-C₁₀ alkenyl, aryl, or a bond to form an optionally substituted 5- or 6-membered ring with R¹¹; and R¹¹ is hydrogen, C₁-C₆ alkyl, C₁-C₁₀ alkenyl, aryl, or a bond to form an optionally substituted 5- or 6-membered ring with R¹⁰.

To perform the reaction, the transition metal complex and ligand may be combined in one or more solvents under inert atmosphere to provide an active catalyst. The inert atmosphere may comprise nitrogen (N₂) gas or argon (Ar) gas, for example.

The molar ratio of the transition metal complex to the ligand may be 1:1 or greater, 1:1.05 or greater, 1:1.10 or greater, 1:1.20 or greater, 1:1.30 or greater, 1:1.40 or less, 1:1.50 or less, 1:1.60 or less, 1:1.70 or less, 1:1.80 or less, 1:1.90 or less, 1:2.00 or less, 1:2.10 or less, 1:2.20 or less, 1:2.50 or less, 1:3 or less, 1:4 or less, 1:5 or less, or any value encompassed by these endpoints.

Suitable solvents may include methanol, ethanol, isopropanol, hexanol, texanol, tetrahydrofuran (THF), toluene, xylene, dioxane, n-butanol, ethyl acetate, dichloromethane (DCM), or diethyl ether (Et₂O), or combinations thereof, for example.

The transition metal complex and ligand may be stirred under inert atmosphere for a

period of time of about 10 minutes or greater, about 20 minutes or greater, about 30 minutes or greater, about 40 minutes or greater, about 50 minutes or greater, about 60 minutes or less, about 70 minutes or less, about 80 minutes or less, about 90 minutes or less, or any value encompassed by these endpoints.

The transition metal complex and ligand may be pre-formed by mixing Rh-precursor and ligand in one or more solvents under inert atmosphere or under an atmosphere of hydrogen or carbon monoxide or a mix of hydrogen and carbon monoxide in any ratio in a pressure range of 1 bar to 100 bar, as described in WO 2006/40096, for example.

The transition metal complex and ligand may be combined at a temperature of about 20° C. or higher, about 30° C. or higher, about 40° C. or higher, about 50° C. or lower, about 60° C. or lower, about 70° C. or lower, about 80° C. or lower, or any value encompassed by these endpoints.

The active catalyst may then be combined with a solution comprising the (2,3)/(4,5) dienone. The solution may further comprise an additional solvent, such as methanol, ethanol, isopropanol, 1-hexanol, 1-decanol, 1-nonanol, texanol (3-hydroxy-2,2,4-trimethylpentyl isobutyrate), tetrahydrofuran (THF), toluene, ethyl acetate, dichloromethane (DCM), MTBE, or diethyl ether (Et₂O), for example.

The solution may further comprise one or more co-solvents, such as an alkyl benzene. Suitable alkyl benzenes may include toluene, ethyl benzene, xylenes, mesitylene, and durene, for example. Further suitable co-solvents may comprise methanol, ethanol, isopropanol, 1-hexanol, 1-decanol, 1-nonanol, texanol (3-hydroxy-2,2,4-trimethylpentyl isobutyrate), tetrahydrofuran (THF), dioxane, n-butanol, ethyl acetate, or diethyl ether (Et20), for example. Co-solvents are most preferably used in an amount of about 5 wt. % or greater, about 10 wt. % or greater, about 15 wt. % or greater, about 20 wt. % or greater, about 25 wt. % or greater, about 30 wt. % or greater, about 40 wt. % or greater, about 45 wt. % or less, about 50 wt. % or less, about 55 wt. % or less, about 60 wt. % or less, about 65 wt. % or less, about 70 wt. % or less, about 75 wt. % or less, about 80 wt. % or less, or any value encompassed by these endpoints, as a percentage of the complete reaction mass.

The amount of the (2,3)/(4,5) dienone in the reaction may be about 5% or greater, about 10% or greater, about 15% or greater, about 20% or greater, about 25% or greater, about 30% or greater, about 35% or greater, about 40% or greater, about 45% or greater, about 50% or less, about 55% or less, about 60% or less, about 65% or less, about 70% or less, about 75% or less, about 80% or less, about 85% or less, about 90% or less, about 95% or less, about 99% or less, about 99.9% or less, or any value encompassed by these endpoints. Preferably, the concentration is about 10% to about 80%, as a percentage of the total reaction mixture.

Following the addition of the active catalyst to the (2,3)/(4,5) dienone solution, the nitrogen (N₂) may be replaced by hydrogen (H₂) by charging to a pressure between 1 and 100 bar. The pressure may then be carefully released, and the process is repeated twice more.

The reaction may be performed under the hydrogen (H₂) atmosphere. The pressure of the hydrogen atmosphere may be about 1 bar or greater, about 5 bar or greater, about 10 bar or greater, about 20 bar or greater, about 30 bar or greater, about 40 bar of greater, about 50 bar or less, about 60 bar or less, about 70 bar or less, about 80 bar or less, about 90 bar or less, about 100 bar or less, or any value encompassing these endpoints.

The hydrogen atmosphere may further comprise carbon monoxide (CO). The carbon monoxide may be present in an amount of about 1 ppm or greater, about 5 ppm or greater, about 10 ppm or greater, about 50 ppm or greater, about 100 ppm or greater, about 200 ppm or greater, about 500 ppm or greater, about 700 ppm or greater, about 1000 ppm or less, about 1200 ppm or less, about 1500 ppm or less, about 1700 ppm or less, about 2000 ppm or less, or any value encompassed by these endpoints.

The reaction may be performed at a temperature of about 10° C. to about 100° C., for example 10° C. or greater, 20° C. or greater, about 30° C. or greater, about 40° C. or greater, about 50° C. or greater, about 60° C. or less, about 70° C. or less, about 80° C. or less, about 90° C. or less, about, or any value encompassed by these endpoints.

The reaction may be stirred for a period of time of about 1 hour or longer, about 2 hours or longer, about 3 hours or longer, about 5 hours or longer, about 10 hours or longer, about 15 hours or longer, about 20 hours or longer, about 24 hours or longer, about 30 hours or less, about 35 hours or less, about 40 hours or less, about 45 hours or less, about 48 hours or less, or any value encompassed by these endpoints.

The reaction may be performed discontinuously or semicontinuously as well as continuously and is suitable in particular for reactions on an industrial scale.

The obtained product can be separated from the catalyst by known procedures such as distillation under reduced pressure. The remaining catalyst can be reused.

### EXAMPLES

Unless otherwise noted, all reactions were conducted under inert atmosphere in a N₂-filled glovebox. All glassware was oven-dried prior to use. Pseudoionone (technical, mixture of isomers, ≥95% by GC and ¹H NMR), Ru(COD)(met)₂, and anhydrous methanol were purchased from Sigma-Aldrich. Rh(CO)₂ (acac) was synthesized using the literature procedure (Inorganic Synthesis, 2004, 34, 128). Ligands were purchased from Sigma-Aldrich or Strem Chemicals, Combi-blocks, Alfa Aesar, or Acros (xantphos CAS: 161265-03-8, dpephos CAS: 166330-10-5, dppm CAS: 2071-20-7, dppe CAS: 1663-45-2, dppp CAS: 6737-42-4, dppb CAS: 7688-25-7, dppf CAS: 12150-46-8, binap CAS: 98327-87-8, spanphos CAS: 556797-94-5, P(OPh)₃ CAS: 101-02-0, P(OMe)₃ CAS: 121-45-9, monophos CAS: 252288-04-3, PPh₃ CAS: 603-35-0, P (4-OMeC₆H₄)₃ CAS: 855-38-9, P (3,5-CF₃—C₆H₃)₃P CAS: 175136-62-6, PPhMe₂ 672-66-2, PCy₃ CAS: 2622-14-2, SPhos CAS 657408-07-6, dippf CAS 97239-80-0). Parr high-pressure reactors (Series 4750 vessels with split ring closure) were used for hydrogenations. Unless otherwise noted, yields and conversions were determined by gas chromatography with durene as the internal standard using either Agilent J&W HP-5 or DB-5 MS columns (30 m).

### Example 1: General Procedure for Rh-Catalyzed Hydrogenation of Pseudoionone, 0.2 mmol Scale

Pseudoionone was reduced according to Scheme 2, below.

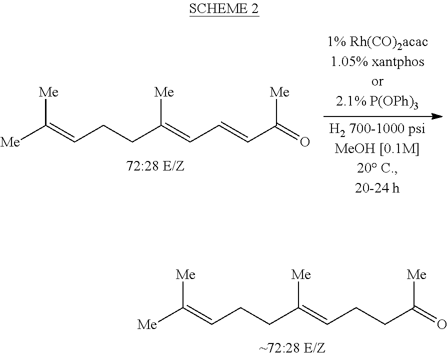 [SMILES: CC(=O)/C=C/C=C(\C)CCC=C(C)C ; CC(=O)CC/C=C(\C)CCC=C(C)C]

In a glovebox filled with N2, a stock solution was made by mixing Rh(CO)₂(acac) (0.004 mmol, 1.0 mg) with xantphos (0.0042 mmol, 2.4 mg) in a 1:1.05 molar ratio in methanol (MeOH) (2 mL) at room temperature for 30 min. An aliquot of the catalyst solution (1.0 mL, 0.002 mmol) was transferred into the vial (1-dram) charged with pseudoionone (72% E, 0.2 mmol, 38.4 mg) and durene (5-10 mg) in MeOH (1.0 mL). A stir-bar was added into the mixture, the vial was sealed with a PTFE-line cap. The PTFE-line cap was pierced with an 18-gauge needle, then the vial was placed into a high-pressure reactor. The high-pressure reactor was sealed and taken out of the glovebox. The N₂ atmosphere of the reactor was replaced by H₂ by charging to 600-800 psi, then carefully releasing the pressure, and repeating this process twice more. The reaction mixture was stirred under corresponding H₂ pressure (700-1000 psi). Pressures as low as 100 psi could be used to obtain similar results.

To determine crude yields, upon completion of the reaction, a small aliquot (about 10 uL) of the reaction mixture was removed, and added to a gas chromatography (GC) vial charged with ethyl acetate (EtOAc), then analyzed by GC to determine conversion and yield, >99% conv. and 97% yield (72% E, 28% Z). [GC conditions: HP-5 or DB-5 MS, 100-300° C., 9 min; retention times on HP-5: pseudoionone: t_{(3E,5Z)}=3.01 min, t_{(3E,5E)}=3.24 min; geranylacetone: t_{(5Z)}=2.57 min, t_{(5E)}=2.65 min. Retention times on DB-5 MS: pseudoionone: t_{(3E,5Z)}=3.21 min, t_{(3E,5E)}=3.46 min; geranylacetone: t_{(5Z)}=2.74 min, t_{(5E)}=2.830 min. Molar response factors compared to durene internal standard: psuedoionone 1.1; geranylacetone: 1.2.

Under the same conditions described above, three other catalysts were used. The catalysts and results are shown below in Table 1.

TABLE 1

| | | | % | % |
|---|---|---|---|---|
| Exp. | Catalyst | Ligand | Conversion | Yield |
| 1.1 | Rh(CO)₂(acac) 1 mol % | P(OPh)₃ 2.1 mol % | >99 | 97 |
| 1.2 | Rh(CO)₂(acac) 0.25 mol % | P(OPh)₃ 0.53 mol % | 28 | 48 |
| 1.3 | Rh(CO)₂(acac) 0.25 mol % | xantphos 0.26 mol % | 99 | 94 |

### Example 2: Reduced Catalyst Loading at Higher Temperature

In this Example, pseudoionone (72% E, 0.2 mmol, 38.4 mg) was selectively reduced using the same 0.2 mmol scale general procedure described above. The catalyst was 0.25 mol % Rh(CO)₂(acac) with 0.53 mol % P(OPh)₃. The reaction was stirred at 50° C. for 24 h. Yields were determined as described above, showing 92% yield and >99% conversion.

### Example 3: Rh-Catalyzed Hydrogenation of Pseudoionone, 1 mmol Scale

In a glovebox filled with N₂, a stock solution was made by mixing Rh(CO)₂(acac) (0.011 mmol, 2.8 mg) with xantphos (0.012 mmol, 6.7 mg) in a 1:1.05 molar ratio in MeOH (5.5 mL) at room temperature for 30 min. The catalyst solution (5 mL, 0.01 mmol) was transferred into the 4-dram vial charged with pseudoionone (72% E, 1 mmol, 192.3 mg) and durene (25 mg) in MeOH (5 mL). A stir-bar was added into the mixture, the vial was sealed with a PTFE-line cap. The PTFE-line cap was pierced with five 18-gauge needles, then the vial was placed into a high-pressure reactor. Alternatively, an 8-dram vial without a cap may be used.

The high-pressure reactor was sealed taken out of the glovebox. The N₂ atmosphere of the reactor was replaced by H₂ by charging to 600-800 psi, then carefully releasing the pressure, this process was repeated two more times. The reaction mixture was stirred under the corresponding H₂ pressure (1000 psi).

To determine crude yields, upon completion of the reaction, a small aliquot (about 10 uL) of the reaction mixture was removed and added to a GC vial charged with EtOAc, then analyzed by GC to determine conversion and yield as described above, >99% conversion and 96% yield (72% E, 28% Z).

### Example 4: Ligand Screen

Using the procedure described in Example 1 (yields and conversion by GC), pseudoionone (72% E, 0.2 mmol, 38.4 mg) was treated with Rh(CO)₂acac (1 mol %) in conjunction with various bisphosphine and phosphine ligands. In each case, the reaction was conducted under H₂ at 1000 psi at 20° C. at a concentration of 0.1M in MeOH, as shown in Scheme 3.

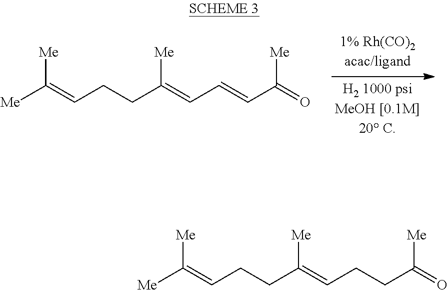 [SMILES: CC(=O)/C=C/C=C(\C)CCC=C(C)C ; CC(=O)CC/C=C(\C)CCC=C(C)C]

The ligands tested, along with percent yields and percent conversions, are shown below in Table 2. Unless otherwise noted, the ratio of rhodium complex to ligand was 1:1 for bisphosphines and 1:2 for monophosphines.

TABLE 2

| | | L | Rh | % | |
|---|---|---|---|---|---|
| Exp. | Ligand | (mol %) | (mol %) | Yield | % Conversion |
| | Bisphosphines | | | | |
| 4.1 | xantphos | 1 | 1 | 95 | >95 |
| 4.2* | xantphos | 0.02 | 0.02 | 94 | >99 |
| 4.3 | DPEphos | 1 | 1 | 91 | >99 |
| 4.4 | DBFphos | 1 | 1 | 3 | 17 |
| 4.5 | dppm | 1 | 1 | 0 | 0 |
| 4.6 | dppe | 1 | 1 | <1 | <1 |
| 4.7 | dppp | 1 | 1 | 64 | >99 |
| 4.8 | dppb | 1 | 1 | 78 | 79 |
| 4.9 | dppf | 1 | 1 | 89 | >99 |
| 4.10 | binap | 1 | 1 | 13 | 23 |
| 4.11 | spanphos | 1 | 1 | 94 | >99 |
| 4.12 | xantphos | 2.1 | 1 | 73 | 74 |
| | Monophosphines | | | | |
| 4.13 | P(OPh)₃ | 2 | 1 | 91 | >99 |
| 4.14* | P(OPh)₃ | 0.04 | 0.02 | 21 | 58 |
| 4.15 | P(OMe)₃ | 2 | 1 | 4 | 26 |
| 4.16 | monophos | 2 | 1 | 87 | >99 |
| 4.17 | PPh₃ | 2 | 1 | 12 | 13 |
| 4.18 | P(4-OMe-C₆H₄)₃ | 2 | 1 | 24 | 24 |
| 4.19 | P(3,5-CF₃-C₆H₃)₃ | 2 | 1 | 74 | 75 |
| 4.20 | PPhMe₂ | 2 | 1 | 43 | 47 |
| 4.21 | PCy₃ | 2 | 1 | 0 | 0 |
| 4.22 | SPhos | 2 | 1 | 7 | 18 |

*reaction temperature at 60° C.

### Example 5: Reaction Condition Screen

Using the procedure described in Example 1, pseudoionone (72% E, 0.2 mmol, 38.4 mg) was treated with Rh(CO)₂acac (1 mol %) and xantphos (1%) as shown in Scheme 4.

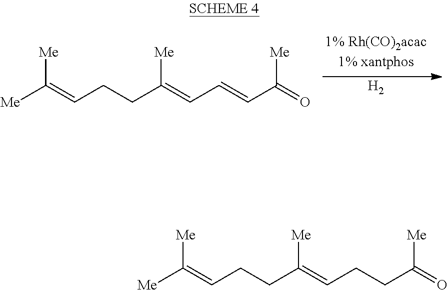 [SMILES: CC(=O)/C=C/C=C(\C)CCC=C(C)C ; "H2" ; CC(=O)CC/C=C(\C)CCC=C(C)C]

Various solvents, pressures, temperatures, and reaction times were tested. The conditions and results are shown below in Table 3.

TABLE 3

| | | | | | | % | |
|---|---|---|---|---|---|---|---|
| | | Pressure, | Temp., | Atmo- | Time, | Con- | % |
| Exp. | Solvent | psi | ° C. | sphere | h | version | Yield |
| 5.1 | MeOH | 1000 | 20 | H₂ | 3 | >99 | 87 |
| 5.2 | MeOH | 200 | 20 | H₂ | 24 | >99 | 90 |
| 5.3 | MeOH | 200 | 70 | H₂ | 3 | >99 | 96 |
| 5.4 | MeOH | 15 | 20 | H₂ | 24 | 84 | 63 |
| 5.5 | Isopropanol | 1000 | 20 | H₂ | 3 | >99 | 92 |
| 5.6 | EtOAc | 1000 | 20 | H₂ | 16 | >99 | 76 |
| 5.7 | THF | 1000 | 20 | H₂ | 17 | >99 | 80 |
| 5.8 | CH₂Cl₂ | 1000 | 20 | H₂ | 4 | 99 | 73 |
| 5.9 | MeOH* | 1000 | 20 | H₂ | 20 | 17 | 2 |
| 5.10 | MeOH | 1000 | 20 | HCO₂H/ | 20 | 39 | 21 |
| | | | | NEt₃** | | | |

*In this run, no ligand was used.
**5:2; 2 equivalents of HCO₂H

### Example 6: Catalyst Screen

Using the procedure described in Example 1, pseudoionone (72% E, 0.2 mmol, 38.4 mg) was treated with a transition metal complex in an amount of 1 mol % and xantphos (1%), as shown in Scheme 5.

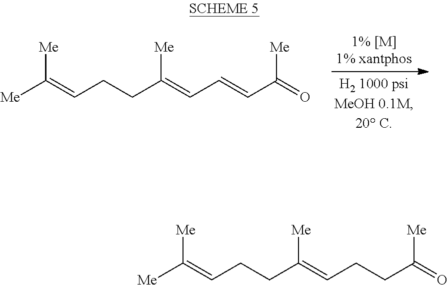 [SMILES: CC(=O)/C=C/C=C(\C)CCC=C(C)C ; CC(=O)CC/C=C(\C)CCC=C(C)C]

In each case, the reaction pressure was 1000 psi under H2 atmosphere; the reaction concentration was 0.1 M in methanol; and the reaction temperature was 20° C. Various transition metal complexes were tested, as shown in Table 4 along with percent conversion and percent yield.

TABLE 4

| Exp. | Metal Complex | % Conversion | % Yield |
|---|---|---|---|
| 6.1 | [Rh(COD)Cl]₂ | >99 | 0 |
| 6.2 | [Rh(ethylene)₂Cl]₂ | 91 | 6 |
| 6.3 | [Rh](COD)₂]BF₄ | 54 | 21 |
| 6.4 | RhCl(PPh₃)₃* | 0 | 0 |
| 6.5 | Ir(CO)₂acac** | 0 | 0 |
| 6.6 | [Ir(COD)Cl]₂ | 33 | 9 |
| 6.7 | Ru(COD)(methallyl)₂** | >99 | 23 |

*no added ligand
**70° C.

### Example 7: Ruthenium Catalyzed Hydrogenation of Pseudoionone

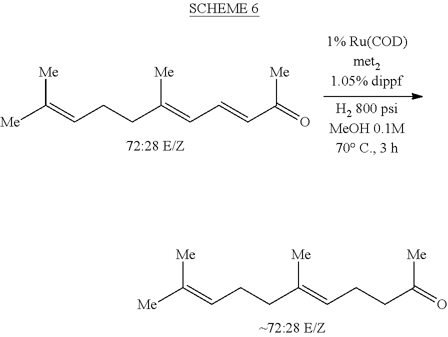 [SMILES: CC(=O)/C=C/C=C(\C)CCC=C(C)C ; CC(=O)CC/C=C(\C)CCC=C(C)C]

The general procedure as described in Example 1 was used to conduct Ru-catalyzed hydrogenations (Ru-Precursor and Ligand replacing the Rh-Precursor and Ligand). The reaction mixture was stirred at 70° C. under H₂ atmosphere at a pressure of 800 psi. To determine crude yields, upon completion of the reaction, a small aliquot (about 10 uL) of the reaction mixture was removed and added to a GC-vial charged with EtOAc, then analyzed by GC to determine conversion and yield as described above, (dippf: >99% conversion and 92% yield, 72% E).

### Example 8: Optimization of Ruthenium Catalyzed Hydrogenation of Pseudoionone

As shown in Scheme 7, pseudoionone was reduced using a ruthenium complex and a variety of ligands under H2 atmosphere at 800 psi. The reaction concentration was 0.2 M in methanol. The reaction was stirred for 2-3 hours at 70° C.-75° C.

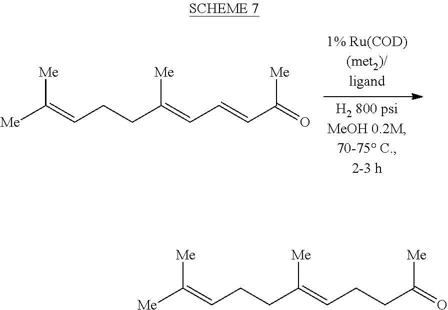 [SMILES: CC(=O)/C=C/C=C(\C)CCC=C(C)C ; CC(=O)CC/C=C(\C)CCC=C(C)C]

The different ligands, along with percent conversion and percent yield, are shown below in Table 5.

TABLE 5

| | | L:Ru | | |
|---|---|---|---|---|
| Exp. | Ligand | (molar ratio) | % Conversion | % Yield |
| 8.1 | None | | 100 | 0 |
| 8.2 | PPh₃ | 2:1 | 4 | 21 |
| 8.3 | PPhMe₂ | 2:1 | >99 | 79 |
| 8.4 | PCy₃ | 2:1 | >99 | 0 |
| 8.5 | dppe | 1:1 | >99 | 26 |
| 8.6 | dppp | 1:1 | >99 | 39 |
| 8.7 | dppb | 1:1 | >99 | 48 |
| 8.8 | dppf | 1:1 | 23 | 13 |
| 8.9 | xantphos | 1:1 | >99 | 23 |
| 8.10 | dippf | 1:1 | 99 | 95 |

As a further variation, two ligands were tested at a 1.5-hour reaction time. The results for these further tests are shown below Table 6.

TABLE 6

| | | L:Ru | | |
|---|---|---|---|---|
| Exp. | Ligand | (molar ratio) | % Conversion | % Yield |
| 8.11 | PPhMe₂ | 2:1 | >99 | 83 |
| 8.12 | dippf | 1:1 | 96 | 93 |

### Example 9.1: Reaction following Rh(CO)₂acac/xantphos Catalyst Preformation

Rh(CO)₂acac (43 mg, 0.17 mmol), and xantphos (140 mg, 0.24 mmol) were dissolved in tetrahydrofuran (THF) (27 ml) and stirred in a 100 ml steel autoclave (V2A steel, manufacturer Premex, magnetically coupled gas-dispersion stirrer, 1000 revolutions/mm) at 1160 psi under synthesis gas (H₂/CO=1:1, vol/vol.). The reaction was maintained at 70° C. for 16 h, then cooled to 25° C. and the pressure released. Nitrogen was passed through the solution for two hours. After flushing with nitrogen, pseudoionone (16.2 g, 84.2 mmol) was added to the autoclave via a lock. The reaction pressure was adjusted to 1160 psi with hydrogen and heated to 50° C. Yield and conversion were determined by gas chromatography (RXI-ms column: 20 m×0.18 mm/0.36 μm; 30 min at 100° C. then 35° C./h to 300° C.). After a reaction time of 4 hours, a conversion of 97% was observed, with a 94% yield of geranylacetone.

### Example 9.2: Reaction Following Rh(CO)₂acac/dppe with Catalyst Preformation

Rh(CO)₂acac (43 mg, 0.17 mmol) and dppe (101 mg, 0.25 mmol) were dissolved in texanol (27 ml) and stirred in a 100 ml steel autoclave (V2A steel, manufacturer Premex, magnetically coupled gas-dispersion stirrer, 1000 revolutions/mm) at 1160 psi under synthesis gas (H₂/CO=1:1, vol/vol.). The reaction was maintained at 70° C. for 16 h, then cooled to 25° C. and the pressure released. Nitrogen was passed through the solution for two hours. After flushing with nitrogen, pseudoionone (16.2 g, 84.2 mmol) was added to the autoclave via a lock. The reaction pressure was adjusted to 1160 psi with hydrogen and heated to 50° C. Yield and conversion were determined by gas chromatography (RXI-ms column: 20 m×0.18 mm/0.36 μm; 30 min at 100° C. then 35° C./h to 300° C.). After a reaction time of 4 hours, a conversion of >98% was observed, with a 97% yield of geranylacetone.

### Example 9.3: Reaction Following Rh(CO)₂acac/PPh₃ with Catalyst Preformation

Rh(CO)₂acac (43 mg, 0.17 mmol) and PPh₃ (135 mg, 0.52 mmol) were dissolved in THF (30 ml) and stirred in a 100 ml steel autoclave (V2A steel, manufacturer Premex, magnetically coupled gas-dispersion stirrer, 1000 revolutions/mm) at 1160 psi under synthesis gas (H₂/CO=1:1, vol/vol.). The reaction was maintained at 70° C. for 16 h, then cooled to 25° C. and the pressure released. Nitrogen was passed through the solution for two hours. After flushing with nitrogen, pseudoionone (16.2 g, 84.2 mmol) was added to the autoclave via a lock. The reaction pressure was adjusted to 1160 psi with hydrogen and heated to 50° C. Yield and conversion were determined by gas chromatography (RXI-ms column: 20 m×0.18 mm/0.36 μm; 30 min at 100° C. then 35° C./h to 300° C.). After a reaction time of 20 hours, a conversion of >98% was observed, with a 97% yield of geranylacetone.

### Example 10: Reaction Following Rh(CO)₂acac/P(OPh)₃ Catalyst Preformation

Rh(CO)₂acac (43 mg, 0.17 mmol) and P(OPh)₃ (155 mg, 0.5 mmol) were dissolved in tetrahydrofuran (THF) (27 ml) and stirred in a 100 ml steel autoclave (V2A steel, manufacturer Premex, magnetically coupled gas-dispersion stirrer, 1000 revolutions/mm) at 1160 psi under synthesis gas (H₂/CO=1:1, vol/vol.). The reaction was maintained at 70° C. for 16 h, then cooled to 25° C. and the pressure released. Nitrogen was then passed through the solution for two hours. After flushing with nitrogen, pseudoionone (15.2 g, 84.2 mmol) was added to the autoclave via a lock. The reaction pressure was adjusted to 1160 psi with hydrogen and heated to 50° C. Yield and conversion were determined by gas chromatography. After a reaction time of 20 hours, a conversion of 99% was observed with a 91% yield of geranylacetone.

### Example 11: Scope of Rh(CO)₂acac/xantphos Catalyzed Reduction

In this Example, Rh(CO)₂acac/xantphos (L: Rh=1:1, MeOH, 1 mol % Rh) was used as the catalyst to reduce a variety of substrates. In each case, the reaction was conducted under an H₂ atmosphere at 1000 psi. The substrates and conditions for each reaction, along with percent conversion, percent yield, and isolated yield are shown below in Table 7. Reactions were run according to the general procedure described in Example 1 with variations listed in table 7, for isolation protocols for the compounds in Exp. 11.1-11.9 (Compounds 2-9) see section Experimental Data.

TABLE 7

| | | | | | | | | % |
|---|---|---|---|---|---|---|---|---|
| | | | Temp. | Time | Scale | % Con- | % | Isolated |
| Exp. | Starting Material | Product | ° C. | h | mmol | version* | Yield* | Yield |
| 11.1 | 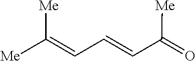 | 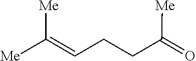 | 20 | 4 | 1.0 | >98 | 90 | 40 |
| 11.2 | 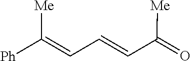 [SMILES: CC(=O)/C=C/C=C(\C)c1ccccc1] | 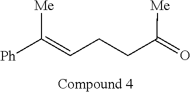 [SMILES: CC(=O)CC/C=C(\C)c1ccccc1] | 20 | 21 | 0.1 | >98 | 95 | 85 |
| 11.3 | 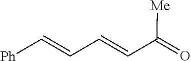 [SMILES: CC(=O)/C=C/C=C/c1ccccc1] | 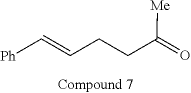 [SMILES: CC(=O)CC/C=C/c1ccccc1] | 20 | 0.5 | 0.1 | >98 | 84 | 83 |
| 11.4 | 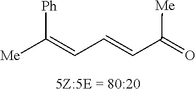 [SMILES: CC(=O)/C=C/C=C(/C)c1ccccc1] | 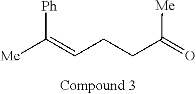 [SMILES: CC(=O)CC/C=C(/C)c1ccccc1] | 20 | 21 | 0.1 | >98 | 93 (5Z:5E = 87:13) | 77 (5Z:5E > 95:5) |
| 11.5 | 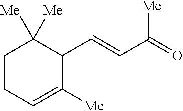 [SMILES: CC(=O)/C=C/C1C(C)=CCCC1(C)C] | 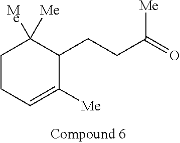 [SMILES: CC(=O)CCC1C(C)=CCCC1(C)C] | 40 | 21 | 0.2 | >98 | >95 | 99 |
| 11.6 | 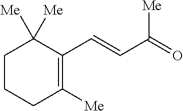 [SMILES: CC(=O)/C=C/C1=C(C)CCCC1(C)C] | 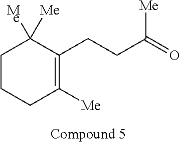 [SMILES: CC(=O)CCC1=C(C)CCCC1(C)C] | 40 | 21 | 0.2 | >98 | >95 | 99 |
| 11.7 | 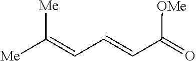 | 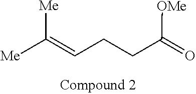 | 50 | 16 | 1.0 | >98 | 75 | nd** |
| 11.8 | 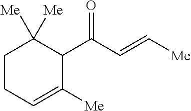 [SMILES: C/C=C/C(=O)C1C(C)=CCCC1(C)C] | 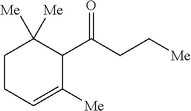 [SMILES: CCCC(=O)C1C(C)=CCCC1(C)C] | 20 | 3 | 0.1 | >98 | >92 | 99 |
| 11.9 | 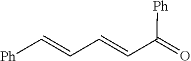 [SMILES: O=C(/C=C/C=C/c1ccccc1)c1ccccc1] | 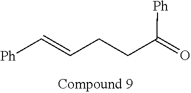 [SMILES: O=C(CC/C=C/c1ccccc1)c1ccccc1] | 20 | 4 | 0.1 | 85 | 84 | 84 |

*yield and conversion determined by ¹H NMR
**nd = not determined

### Example 12: Scope of Rh(CO)₂acac/P (OPh); Catalyzed Reduction

In this Example, Rh(CO)₂acac/P(OPh)₃ (L: Rh=2:1, MeOH, 1 mol % Rh) was used as the catalyst to reduce a variety of substrates. In each case, the reaction was conducted under an H₂ atmosphere at 1000 psi. The substrates and conditions for each reaction, along with percent conversion, and percent yield are shown below in Table 8. Reactions were run according to the general procedure described in Example 1 with variations listed in table 8.

TABLE 8

| | | | Temp. | Time | Scale | % Con- | % |
|---|---|---|---|---|---|---|---|
| Exp. | Starting Material | Product | ° C. | h | mmol | version* | Yield* |
| 12.1 | 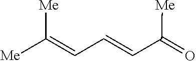 | 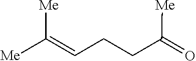 | 20 | 21 | 0.1 | 49 | 45 |
| 12.2 | 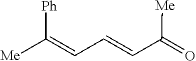 [SMILES: CC(=O)/C=C/C=C(/C)c1ccccc1] | 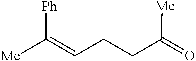 [SMILES: CC(=O)CC/C=C(/C)c1ccccc1] | 20 | 21 | 0.1 | 73 (5Z:5E = 89:11) | 50 (5Z:5E = 78:22) |
| 12.3 | 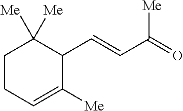 [SMILES: CC(=O)/C=C/C1C(C)=CCCC1(C)C] | 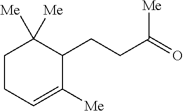 [SMILES: CC(=O)CCC1C(C)=CCCC1(C)C] | 40 | 21 | 0.1 | 64 | 61 |
| 12.4 | 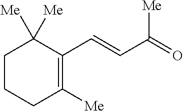 [SMILES: CC(=O)/C=C/C1=C(C)CCCC1(C)C] | 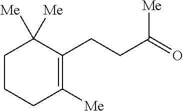 [SMILES: CC(=O)CCC1=C(C)CCCC1(C)C] | 40 | 21 | 0.1 | 26 | 25 |
| 12.5 | 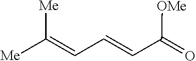 | 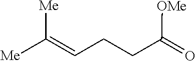 | 45 | 21 | 0.1 | 82 | 40 |

*yield and conversion determined by ¹H NMR

### Experimental Data for Examples 3 and 11 and Compounds in Table 7

Results of NMR and high-resolution mass spectrometry (HRMS) analysis for the compounds of Examples 3 and 11, along with certain purification conditions, are shown below.

### Compound 1, Example 3

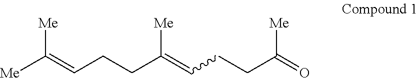 [SMILES: CC(=O)CCC=C(C)CCC=C(C)C]

Compound 1 was prepared from pseudoionone (192.3 mg, 1.0 mmol) according to procedure described in Example 3. The reaction proceeded to greater than 98% conversion and 90% yield (72:28 E/Z ratio) as determined by calibrated GC using durene as the internal standard (ISTD). The product was isolated in 88% yield as a colourless oil after purification by flash column chromatography (2% to 18% EtOAc in hexanes) as a mixture of geometric isomers (5E:5Z=72:28). ¹H NMR (CDCl₃, 500 MHz) δ 5.13-5.04; (m, 2H), 2.44; (dt J=7.4, 7.1 Hz, 2H), 2.30-2.22; (m, 2H), 2.13; (s, 3H), 2.09-2.01; (m, 2H), 2.00-1.94; (m, 2H), 1.67; (s, 3H), 1.61; (s, 3H), 1.59; (s, 3H); ¹³C NMR (CDCl₃, 125 MHz, major, E-prod) δ 208.9, 136.4, 131.5, 124.2, 122.6, 43.8, 39.7, 30.0, 26.7, 25.7, 22.5, 17.7, 16.0; ¹³C NMR (CDCl₃, 125 MHz, minor, Z-prod) δ 208.8, 136.5, 131.7, 124.2, 123.4, 44.1, 31.9, 29.9, 26.5, 25.8, 23.4, 22.3, 17.7; HRMS (EI): calcd for C₁₃H₂₂O [M]⁺ 194.1665. Found 194.1672.

### Compound 2, Example 11.7

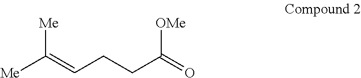

Compound 2 was prepared from the corresponding diene (29.3 mg, 0.21 mmol) according to the general procedure described in Example 1 at 50° C. The reaction was checked by ¹H NMR to confirm full starting material consumption after 16 hours. The product was isolated as a 75:25 mixture of product to over-reduction. ¹H NMR (CDCl₃, 500 MHz) δ 5.11-5.06; (m, 1H), 3.67; (s, 3H), 2.36-2.27; (m, 4H), 1.68; (s, 3H), 1.62; (s, 3H); ¹³C NMR (CDCl₃, 125 MHz) δ 173.9, 133.2, 122.4, 51.5, 34.3, 25.7, 23.7, 17.7; HRMS (EI): calcd for C₈H₁₄O₂ [M]⁺ 142.09938. Found 142.09963. HRMS (EI): calcd for C₈H₁₄O₂ [M]⁺ 142.0988. Found 142.0996.

### Compound 3, Example 11.4

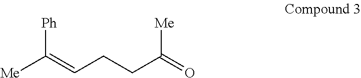 [SMILES: CC(=O)CC/C=C(/C)c1ccccc1]

Compound 3 was prepared from the corresponding diene (18 mg, 0.097 mmol, about 80:20 Z/E) according to the general procedure described in Example 1. The reaction proceeded to greater than 98% conversion and 93% yield (5Z:5E=83:17) as determined by calibrated ¹H NMR with durene as ISTD. The product (5Z isomer only) was isolated in 77% yield after purification by thin-layer chromatography (10% EtOAc in hexanes). ¹H NMR (CDCl₃, 500 MHz) δ 7.35-7.31; (m, 2H), 7.24; (tt, J=7.4, 1.4 Hz, 1H), 7.18-7.15; (m, 2H), 5.41; (tq, J=7.4, 1.4 Hz, 1H), 2.43; (t, J=7.4 Hz, 2H), 2.25; (q, J=7.1 Hz, 2H), 2.07; (s, 3H), 2.02-2.01; (m, 3H); ¹³C NMR (CDCl₃, 125 MHz) δ 208.5, 141.8, 137.7, 128.2, 127.9, 126.7, 125.5, 44.0, 29.8, 25.6, 23.6; HRMS (EI): calcd for C₁₃H₁₆O [M]⁺ 188.1196. Found 188.1205.

### Compound 4, Example 11.2

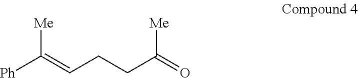 [SMILES: CC(=O)CC/C=C(\C)c1ccccc1]

Compound 4 was prepared from the corresponding diene (17.8 mg, 0.096 mmol) according to general procedure described in Example 1. The reaction proceeded to greater than 98% conversion and 95% yield as determined by calibrated ¹H NMR using durene as ISTD. Product was isolated in 85% (5E isomer only) after purification by thin-layer chromatography (10% EtOAc in hexanes). ¹H NMR (CDCl₃, 500 MHz) δ 7.37-7.34; (m, 2H), 7.32-7.28; (m, 2H), 7.45-7.21; (m, 1H), 5.71; (tq, J=7.2, 1.4 Hz, 1H), 2.60, (t, J=7.4 Hz, 2H), 2.48; (q, J=7.4 Hz, 2H), 2.17; (s, 3H), 2.06-2.05; (m, 3H); ¹³C NMR (CDCl₃, 125 MHz) δ 208.4, 143.7, 136.0, 128.2, 126.7, 126.4, 125.7, 43.4, 30.0, 23.2, 15.9; HRMS (EI): calcd for C₁₃H₁₆O [M]⁺ 188.1196. Found 188.1202.

### Compound 5, Example 11.6

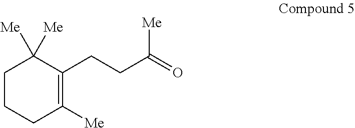 [SMILES: CC(=O)CCC1=C(C)CCCC1(C)C]

Compound 5 was prepared from β-ionone (39.5 mg, 0.21 mmol) according to the general procedure described in Example 1 at 40° C. The reaction proceeded to greater than 98% conversion with no side products observed by ¹H NMR. The product was isolated in 99% after purification through a silica plug (25% EtOAc in hexanes). ¹H NMR (CDCl₃, 500 MHz) δ 2.52-2.47; (m, 2H), 2.28-2.23; (m, 2H), 2.14; (s, 3H), 1.90; (t, J=6.4 Hz, 2H), 1.59-1.53; (m, 5H), 1.43-1.39; (m, 2H), 0.97; (s, 6H); ¹³C NMR (CDCl₃, 125 MHz) δ 209.1, 136.0, 127.8, 44.6, 39.8, 35.1, 32.8, 29.8, 28.5, 22.3, 19.8, 19.5; HRMS (EI): calcd for C₁₃H₂₂O [M]⁺ 194.1665. Found 194.1672.

### Compound 6, Example 11.5

Compound 6 was prepared from α-ionone (38.5 mg, 0.20 mmol) according to the general procedure described in Example 1 at 40° C. The reaction proceeded to greater than 98% conversion with no side products observed by ¹H NMR. The product was isolated in 99% after purification through a silica plug (25% EtOAc in hexanes). ¹H NMR (CDCl₃, 500 MHz) δ 5.33; (m, 1H), 2.461; (dd, J=12.8, 9.9 Hz, 1H), 2.460; (d, J=10.2 Hz, 1H), 2.13; (s, 3H), 1.99-1.94; (m, 2H), 1.76; (ddt, J=14.9, 10.4, 5.6 Hz, 1H), 1.66; (dtd, J=2.1, 1.8, 0.5 Hz, 3H), 1.60; (dddd, J=19.0, 9.9, 6.7, 4.5 Hz, 1H), 1.47; (dd, J=4.5, 4.5 Hz, 1H), 1.40; (ddd, J=17.0, 9.4, 7.7 Hz, 1H), 1.13; (m, 1H), 0.91; (s, 3H), 0.87; (s, 3H); ¹³C NMR (CDCl₃, 125 MHZ) δ 209.2, 135.6, 121.1, 48.5, 43.8, 32.6, 31.6, 30.0, 27.7, 27.6, 24.4, 23.6, 23.0; HRMS (EI): calcd for C₁₃H₂₂O [M]⁺ 194.1665. Found 194.1670.

### Compound 7, Example 11.3

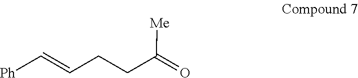 [SMILES: CC(=O)CC/C=C/c1ccccc1]

Compound 7 was prepared according to the general procedure described in Example 1 (0.1 mmol scale procedure) from the corresponding diene (17.2 mg, 0.1 mmol), 0.5 h. ¹H NMR diene conversion: >98%, crude yield: 84%. Isolated in 83% yield by silica column chromatography (10% EtOAc in hexanes). ¹H NMR (CDCl₃, 700 MHZ) δ 7.33-7.28; (m, 4H), 7.21-7.19; (m, 1H), 6.41; (d, J =15.6 Hz, 1H), 6.20; (dt, J=16.0, 7.0 Hz, 1H), 2.61; (t, J=7.6 Hz, 2H), 2.51-2.49; (m, 2H), 2.17; (s, 3H); ¹³C NMR (CDCl₃, 125 MHZ) δ 208.1, 137.5, 130.8, 128.9, 128.6, 127.2, 126.1, 43.3, 30.1, 27.2; HRMS (EI): calcd for C₁₂H₁₄O [M]⁺ 174.1039. Found 174.1044.

### Compound 9, Example 11.9

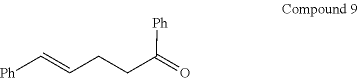 [SMILES: O=C(CC/C=C/c1ccccc1)c1ccccc1]

Compound 9 was prepared according to the general procedure described in Example 1 (0.1 mmol scale procedure) from the corresponding diene (23.4 mg, 0.1 mmol), 4 h. 1H NMR diene conversion: 85%, crude yield: 84%. Isolated in 84% yield by silica column chromatography (2% EtOAc in hexanes). ¹H NMR (CDCl₃, 500 MHz) δ 8.00-7.97; (m, 2H), 7.59-7.55; (m, 1H), 7.50-7.45; (m, 2H), 7.36-7.32; (m, 2H), 7.31-7.27; (m, 2H), 7.22-7.18; (m, 1H), 6.47; (d, J=15.8 Hz, 1H), 6.30; (dt, J=15.7, 6.8 Hz, 1H), 3.16; (t, J=7.6 Hz, 2H), 2.69-2.64; (m, 2H); ¹³C NMR (CDCl₃, 125 MHz) δ 199.4, 137.5, 136.9, 133.1, 130.9, 129.2, 128.7, 128.6, 128.1, 127.1, 126.1, 38.3, 27.6; HRMS (EI): calcd for C₁₇H₁₆O [M]⁺ 236.1196. Found 236.1199.

### Comparative Example A

Finally, previously known conditions were tested using pseudoionone as the substrate, as shown below in Scheme 8.

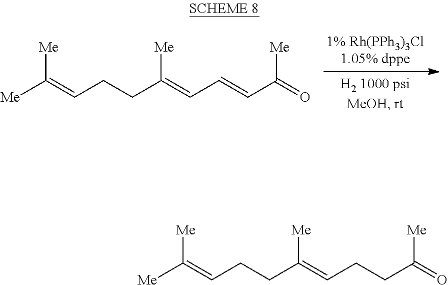 [SMILES: CC(=O)/C=C/C=C(\C)CCC=C(C)C ; CC(=O)CC/C=C(\C)CCC=C(C)C]

The reaction mixture was analyzed at two different time periods, with the results shown below in Table 9.

TABLE 9

| Exp. | Time | Product (%) | SM (% conv.) | Z:E |
|---|---|---|---|---|
| 13.1 | 1 h | 0 | 0 | — |
| 13.2 | 17 h | 15 | 24 | 25:75 |

As can be seen, previously known conditions fail to provide acceptable conversion and selectivity in the case of pseudoionone.

### EMBODIMENTS

Embodiment 1 is a method for selective hydrogenation of dienones, the method comprising: 1) combining a dienone with one or more solvents; 2) adding a catalyst to the mixture of dienone and solvent to provide a reaction mixture; 3) contacting the reaction mixture with an atmosphere comprising hydrogen (H₂); wherein the catalyst comprises one or more transition metals and one or more ligands; and wherein the reaction is performed in the absence of pyridine, pyrazine, quinoline, and quinoxaline.

Embodiment 2 is the method of Embodiment 1, wherein the one or more transition metal is selected from the group comprising ruthenium, rhodium, platinum, palladium, and nickel.

Embodiment 3 is the method of Embodiment 1 or Embodiment 2, wherein the one or more transition metal comprises a rhodium or ruthenium metal complex.

Embodiment 4 is the method of any one of Embodiments 1 to 3, wherein the one or more transition metal comprises Rh(CO)₂acac, Rh(III) acetate, or [Ru(COD)(2-methylallyl)₂].

Embodiment 5 is the method of Embodiment 4, wherein the one or more transition metal comprises Rh(CO₂)acac.

Embodiment 6 is the method of Embodiment 4, wherein the one or more transition metal comprises [Ru(COD)(2-methylallyl)₂].

Embodiment 7 is the method of any one of Embodiments 1 to 6, wherein the one or more ligand is selected from the group comprising 4,5-bis(dipenylphosphino)-9,9-dimethylxanthene (xantphos), bis[(2-diphenylphosphino)phenyl]ether (DPEphos), bis(diphenylphosphino)methane (dppm), 1,2-bis diphenylphosphino)ethane (dppe), 1,3-bis(diphenylphosphino)propane (dppp), 1,4-bis(diphenylphosphino)butane (dppb), 1,1′-bis(diphenylphosphino)ferrocene (dppf), 2,2′-bis(diphenylphosphino)-1,1′-binaphthyl (BINAP), (4,4,4′,4′,6,6′-hexamethyl-3,3′,4,4′-tetrahydro-2,2′-spirobi[[1]benzopyran]-8,8′-diyl)bis(diphenylphosphane) (SPANPhos), triphenyl phosphite (P(OPh)₃), trimethyl phosphite (P(OMe)₃), triethyl phosphite (P(OEt)₃), (3,5-dioxa-4-phosphacyclohepta[2,1-a:3,4-a′]dinaphthalene-4-yl)dimethylamine (MonoPhos), 6,6′-[(3,3′-di-tert-butyl-5,5′-dimethoxy-[1,1′-biphenyl]-2,2′-diyl)bis(oxy)]bis(6H-dibenzo[d,f][1,3,2]dioxaphosphepine) (BiPhePhos), (R,R) Chiraphos, (S,S) Chiraphos, 2-dicyclohexylphosphino-2′,6′-dimethoxybiphenyl (SPhos), triphenylphosphine (PPh₃), tris (4-methoxyphenyl)phosphane, tris(3,5-bis(trifluoromethyl)phenyl)phosphane, 1,1′-bis(diisopropylphosphino)ferrocene (dippf), and methyldiphenylphosphane.

Embodiment 8 is the method of any one of Embodiments 1 to 7, wherein the one or more ligand is selected from the group comprising 4,5-bis(dipenylphosphino)-9,9-dimethylxanthene (xantphos), 1,2-bis(diphenylphosphino)ethane (dppe), (3,5-dioxa-4-phosphacyclohepta[2,1-a:3,4-a′] dinaphthalene-4-yl)dimethylamine (MonoPhos), (R,R) Chiraphos, (S,S) Chiraphos, and triphenylphosphite.

Embodiment 9 is the method of any one of Embodiments 1 to 7, wherein the one or more ligand is selected from the group comprising 1,1′-bis(diisopropylphosphino)ferrocene (dippf) and 1,4-bis(diphenylphosphino)butane (dppb).

Embodiment 10 is the method of any one of Embodiments 1 to 9, wherein the one or more ligands is combined with the transition metal or transition metal complex in a molar ratio of about 1:1 to about 10:1.

Embodiment 11 is the method of any one of Embodiments 1 to 10, wherein the transition metal complex is present in the reaction in an amount of about 0.01 mol % to about 1.0 mol %.

Embodiment 12 is the method of any one of Embodiments 1 to 11, wherein the ligand is present in the reaction in an amount of about 0.01 mol % to about 10.0 mol %.

Embodiment 13 is the method of any one of Embodiments 1 to 12, wherein the one or more solvents are selected from the group consisting of methanol, 1-butanol, 1-propanol, 2-propanol, tetrahydrofuran, toluene, ethyl acetate, and ethanol.

Embodiment 14 is the method of any one of Embodiments 1 to 13, further comprising one or more co-solvents.

Embodiment 15 is the method of Embodiment 14, wherein the co-solvent comprises an alkyl benzene.

Embodiment 16 is the method of any one of Embodiments 1 to 15, wherein the hydrogen atmosphere is at a pressure of about 1 bar to 100 bar, preferably 5 bar to 90 bar, more preferably 10 bar to 80 bar.

Embodiment 17 is the method of Embodiment 16, wherein the hydrogen atmosphere further comprises carbon monoxide in an amount of about 1 ppm to about 2000 ppm.

Embodiment 18 is the method of any one of Embodiments 1 to 17, wherein the dienone is a (2,3)/(4,5) unsaturated dienone of Formula I

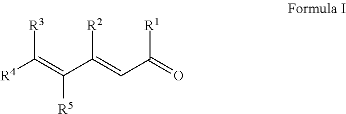

wherein R¹ is C₁-C₆ alkyl, C₁-C₆ alkoxy, or a bond to form an optionally substituted 5- or 6-membered ring with R²; R² is hydrogen, C₁-C₆ alkyl, or a bond to form an optionally substituted 5- or 6-membered ring with R¹; R³ is hydrogen, C₁-C₆ alkyl, C₁-C₁₀ alkenyl, or aryl; R⁴ is hydrogen, C₁-C₆ alkyl, C₁-C₁₀ alkenyl, aryl, or a bond to form an optionally substituted 5- or 6-membered ring with R⁵; and R⁵ is hydrogen, C₁-C₆ alkyl, C₁-C₁₀ alkenyl, aryl, or a bond to form an optionally substituted 5- or 6-membered ring with R⁴.

Embodiment 19 is the method of Embodiment 18, wherein R¹ is C₁-C₆ alkyl or C₁-C₆ alkoxy; R² is hydrogen or C₁-C₆ alkyl; R³ is hydrogen, C₁-C₆ alkyl, C₁-C₁₀ alkenyl, or aryl; and R⁴ is hydrogen, C₁-C₆ alkyl, C₁-C₁₀ alkenyl, or aryl.

Embodiment 20 is the method of Embodiment 18, wherein R¹ is C₁-C₆ alkyl; R² is hydrogen; R³ is C₁-C₆ alkyl; and R⁴ is C₁-C₁₀ alkenyl.

Embodiment 21 is the method of Embodiment 18, wherein the (2,3)/(4,5) unsaturated dienone comprises β-ionone or pseudoionone.

Embodiment 22 is the method of any one of Embodiments 1 to 17, wherein the dienone is a (2,3)/(5,6) unsaturated dienone of Formula II, shown below.

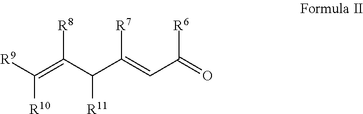

wherein R⁶ is C₁-C₆ alkyl, or C₁-C₆ alkoxy; R⁷ is hydrogen, or C₁-C₆ alkyl; R⁸ is hydrogen, C₁-C₆ alkyl, C₁-C₁₀ alkenyl, or aryl; R⁹ is hydrogen, C₁-C₆ alkyl, C₁-C₁₀ alkenyl, or aryl; R¹⁰ is hydrogen, C₁-C₆ alkyl, C₁-C₁₀ alkenyl, aryl, or a bond to form an optionally substituted 5- or 6-membered ring with R¹¹; and R¹¹ is hydrogen, C₁-C₆ alkyl, C₁-C₁₀ alkenyl, aryl, or a bond to form an optionally substituted 5- or 6-membered ring with R¹⁰.

Embodiment 23 is the method of Embodiment 22, wherein the (2,3)/(5,6) unsaturated dienone comprises α-ionone.

Embodiment 24 is the method of any one of Embodiments 1 to 23, wherein the dienone is monohydrogenated.

Embodiment 25 is the method of any one of Embodiments 1 to 24, wherein the active catalyst comprises Rh(CO)₂acac or Ru(COD)met₂ and one or more of 4,5-bis(dipenylphosphino)-9,9-dimethylxanthene (xantphos), bis[(2-diphenylphosphino)phenyl] ether (DPEphos), bis(diphenylphosphino)methane (dppm), 1,2-bis(diphenylphosphino)ethane (dppe), 1,3-bis(diphenylphosphino)propane (dppp), 1,4-bis(diphenylphosphino)butane (dppb), 1,1′-bis(diphenylphosphino)ferrocene (dppf), 2,2′-bis(diphenylphosphino)-1,1′-binaphthyl (BINAP), (4,4,4′,4′,6,6′-hexamethyl-3,3′,4,4′-tetrahydro-2,2′-spirobi[[1]benzopyran]-8,8′-diyl)bis(diphenylphosphane) (SPANPhos), triphenyl phosphite (P(OPh)₃), trimethyl phosphite (P(OMe)₃), (3,5-dioxa-4-phosphacyclohepta[2,1-a:3,4-a′]dinaphthalene-4-yl) dimethylamine (MonoPhos), 2-dicyclohexylphosphino-2′,6′-dimethoxybiphenyl (SPhos), triphenylphosphine (PPh₃), tris(4-methoxyphenyl)phosphane, tris(3,5-bis(trifluoromethyl)phenyl)phosphane, 1,1′-bis(diisopropylphosphino)ferrocene (dippf), and methyldiphenylphosphane.

Embodiment 26 is the method of any one of Embodiments 1 to 25, wherein the reaction is performed substantially in the absence of pyridine, pyrazine, quinoline, and quinoxaline.

Embodiment 27 method of any one of Embodiments 1 to 26, wherein the catalyst is pre-formed by mixing Rh-precursor and ligand in a solvent under inert atmosphere or under an atmosphere of hydrogen or carbon monoxide or a mix of hydrogen and carbon monoxide in any ratio in a pressure range of 1 bar to 100 bar.

Embodiment 28 is the method of any one of Embodiments 1 to 27, wherein the catalyst is a carbonyl containing Rh-phosphine-catalyst of type L₂Rh(CO)H or L₃Rh(CO)H wherein L is a monodentate phosphine or monodentate phosphite.

Embodiment 29 is the method of any one of Embodiments 1 to 27, wherein the catalyst

is a carbonyl containing Rh-phosphine-catalyst of type L′Rh(CO) or L′Rh(CO)₂H, wherein L′ is a bidentate phosphine or bidentate phosphite).

1-31. (canceled)

32. A process for the selective mono hydrogenation of dienones with hydrogen, the method comprising:
1. combining a dienone with one or more solvents or without solvent;
2. adding a catalyst to the mixture of dienone and solvent or to the pure dienone to provide a reaction mixture;
3. contacting the reaction mixture with an atmosphere comprising hydrogen (H₂);
wherein the catalyst comprises a transition metal and one or more phosphine ligands and/or one or more neutral and/or anionic ligands; and
wherein the reaction is performed in the absence of pyridine, pyrazine, quinoline, and quinoxaline.

33. The process of claim 32, wherein the catalyst comprises a rhodium or ruthenium metal complex.

34. The process of claim 32, wherein the catalyst is formed by reacting a transition metal containing precursor with the ligand to form a transition metal-ligand complex, wherein the metal containing precursor comprises Rh(CO)₂acac, Rh(III)acetate, or [Ru(COD)(2-methylallyl)₂].

35. The method of claim 34, wherein the transition metal containing precursor is Rh(CO)₂acac.

36. The method of claim 34, wherein the transition metal containing precursor is [Ru(COD)(2-methylallyl)₂].

37. The method of claim 32, wherein the one or more ligand is selected from the group consisting of 4,5-bis(dipenylphosphino)-9,9-dimethylxanthene (xantphos), 1,2-bis(diphenylphosphino)ethane (dppe), 1,4-bis(diphenylphosphino)butane (dppb), 1,1′-bis(diphenylphosphino)ferrocene (dppf), (3,5-dioxa-4-phosphacyclohepta[2,1-a:3,4-a′]dinaphthalene-4-yl)dimethylamine (MonoPhos), (R,R) Chiraphos, (S,S) Chiraphos, and triphenylphosphite, and triphenylphosphine (PPh₃).

38. The method of claim 32, wherein the one or more ligand is selected from the group consisting of 1,1′-bis(diisopropylphosphino)ferrocene (dippf) and 1,4-bis(diphenylphosphino)butane (dppb).

39. The method of claim 32, wherein the one or more ligands is combined with the transition metal or transition metal complex in a molar ratio of about 1:1 to about 10:1.

40. The method of claim 32, wherein the transition metal complex is present in the reaction in an amount of about 0.01 mol % to about 1.0 mol %.

41. The method of claim 32, wherein the ligand is present in the reaction in an amount of about 0.01 mol % to about 10.0 mol %.

42. The method of claim 32, wherein the one or more solvents are selected from the group consisting of methanol, 1-butanol, 1-propanol, 2-propanol, tetrahydrofuran, toluene, ethyl acetate, and ethanol.

43. The method of claim 32, further comprising one or more co-solvents.

44. The method of claim 43, wherein the co-solvent comprises an alkyl benzene.

45. The method of claim 32, wherein the atmosphere comprising hydrogen is at a pressure of about 1 bar to 100 bar.

46. The method of claim 32, wherein the reaction is performed at a temperature of about 10° C. to about 100° C.

47. The method of claim 45, wherein the atmosphere comprising hydrogen further comprises carbon monoxide in an amount of about 1 ppm to about 2000 ppm.

48. The method of claim 32, wherein the dienone is a (2,3)/(4,5) unsaturated dienone of Formula I 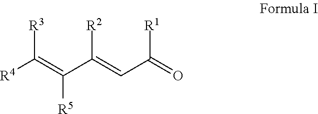
wherein R¹ is C₁-C₆ alkyl, C₁-C₆ alkoxy, or a bond to form an optionally substituted 5- or 6-membered ring with R²;
R² is hydrogen, C₁-C₆ alkyl, or a bond to form an optionally substituted 5- or 6-membered ring with R¹;
R³ is hydrogen, C₁-C₆ alkyl, C₁-C₁₀ alkenyl, or aryl;
R⁴ is hydrogen, C₁-C₆ alkyl, C₁-C₁₀ alkenyl, aryl, or a bond to form an optionally substituted 5- or 6-membered ring with R⁵; and
R⁵ is hydrogen, C₁-C₆ alkyl, C₁-C₁₀ alkenyl, aryl, or a bond to form an optionally substituted 5- or 6-membered ring with R⁴.

49. The method of claim 48, wherein the (2,3)/(4,5) unsaturated dienone comprises β-ionone, 6-methyl-3,5-heptadien-2-one or pseudoionone.

50. The method of claim 32, wherein the dienone is a (2,3)/(5,6) unsaturated dienone of Formula II, shown below. 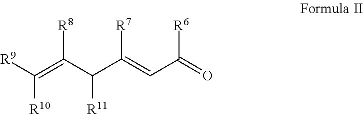
wherein R⁶ is C₁-C₆ alkyl, or C₁-C₆ alkoxy;
R⁷ is hydrogen, or C₁-C₆ alkyl;
R⁸ is hydrogen, C₁-C₆ alkyl, C₁-C₁₀ alkenyl, or aryl;
R⁹ is hydrogen, C₁-C₆ alkyl, C₁-C₁₀ alkenyl, or aryl;
R¹⁰ is hydrogen, C₁-C₆ alkyl, C₁-C₁₀ alkenyl, aryl, or a bond to form an optionally substituted 5- or 6-membered ring with R¹¹; and
R¹¹ is hydrogen, C₁-C₆ alkyl, C₁-C₁₀ alkenyl, aryl, or a bond to form an optionally substituted 5- or 6-membered ring with R¹⁰.

51. The method of claim 50, wherein the (2,3)/(5,6) unsaturated dienone comprises α-ionone.

52. The method of claim 32, wherein the active catalyst comprises Rh(CO)₂acac or Ru(COD)met₂ and one or more of 4,5-bis(dipenylphosphino)-9,9-dimethylxanthene (xantphos), bis[(2-diphenylphosphino)phenyl]ether (DPEphos), bis(diphenylphosphino)methane (dppm), 1,2-bis(diphenylphosphino)ethane (dppe), 1,3-bis(diphenylphosphino)propane (dppp), 1,4-bis(diphenylphosphino)butane (dppb), 1,1′-bis(diphenylphosphino)ferrocene (dppf), 2,2′-bis(diphenylphosphino)-1,1′-binaphthyl (BINAP), (4,4,4′,4′,6,6′-hexamethyl-3,3′,4,4′-tetrahydro-2,2′-spirobi[[1]benzopyran]-8,8′-diyl)bis(diphenylphosphane) (SPANPhos), triphenyl phosphite (P(OPh)₃), trimethyl phosphite (P(OMe)₃), (3,5-dioxa-4-phosphacyclohepta[2,1-a:3,4-a′] dinaphthalene-4-yl) dimethylamine (MonoPhos), 2-dicyclohexylphosphino-2′,6′-dimethoxybiphenyl (SPhos), triphenylphosphine (PPh₃), tris(4-methoxyphenyl)phosphane, tris(3,5-bis(trifluoromethyl)phenyl)phosphane, 1,1′-bis(diisopropylphosphino)ferrocene (dippf), and methyldiphenylphosphane.

53. The method of claim 32, wherein the catalyst is pre-formed by mixing Rh-precursor and ligand in a solvent under inert atmosphere or under an atmosphere of hydrogen or carbon monoxide or a mix of hydrogen and carbon monoxide in any ratio in a pressure range of 1 bar to 100 bar.

54. The method of claim 32, wherein the catalyst is a carbonyl containing Rh-phosphine-catalyst of type L₂Rh(CO)H or L₃Rh(CO)H, wherein L is a monodentate phosphine or monodentate phosphite.
